(19) United States
(12) Patent Application Publication
(10) Pub. No.: US 2016/0376208 A1
(43) Pub. Date: Dec. 29, 2016

(54) Method of Producing Higher Value Hydrocarbons by Isothermal Oxidative Coupling of Methane

(71) Applicant: Sabic Global Technologies, B.V., BERGEN OP ZOOM (NL)

(72) Inventors: Vidya Sagar Reddy SARSANI, Pearland, TX (US); David WEST, Bellaire, TX (US); Aghaddin MAMEDOV, Sugar Land, TX (US); Wugeng LIANG, Richmond, TX (US); James LOWREY, Pearland, TX (US); Istvan LENGYEL, Lake Jackson, TX (US)

(21) Appl. No.: 15/180,955

(22) Filed: Jun. 13, 2016

Related U.S. Application Data
(60) Provisional application No. 62/183,453, filed on Jun. 23, 2015.

(51) Int. Cl. C07C 2/84; C01B 3/50; C01B 3/38

(52) U.S. Cl. CPC C07C 2/84; C01B 3/386; C01B 3/506; C07C 2523/34; C07C 2523/30; C07C 2523/04; C07C 2521/08; C01B 2203/0261; C01B 2203/046; C01B 2203/1041; C01B 2203/1082

(57) ABSTRACT

A method for producing olefins comprising (a) introducing to an isothermal reactor a reactant mixture comprising CH₄ and O₂, wherein the reactor comprises a catalyst bed comprising a catalyst, wherein a catalyst bed temperature is 750-1,000° C., and wherein the reactor has a residence time of 1-100 ms; (b) wherein isothermal conditions minimize hot spots in the bed, thereby decreasing deep oxidation reactions; (c) allowing the reactant mixture to contact the catalyst and react via oxidative coupling of CH₄ reaction to form a product mixture comprising C₂₊ hydrocarbons (olefins and paraffins; C₂ hydrocarbons and C₃ hydrocarbons) and synthesis gas (H₂ and CO), wherein the product mixture has an olefin/paraffin molar ratio of from 0.5:1 to 20:1, and wherein the product mixture has a H₂/CO molar ratio of from 0.2:1 to 2.5:1; (d) recovering the product mixture from the reactor; and (e) recovering C₂ hydrocarbons and/or synthesis gas from the product mixture.

### CROSS-REFERENCE TO RELATED APPLICATIONS

The present application is a non-provisional of and claims priority to U.S. Provisional Patent Application No. 62/183,453 filed Jun. 23, 2015 and entitled “Method for Producing Higher Value Hydrocarbons by Isothermal Oxidative Coupling of Methane,” which application is incorporated by reference herein in its entirety.

### TECHNICAL FIELD

The present disclosure relates to methods of producing hydrocarbons, more specifically methods of producing olefins by oxidative coupling of methane.

### BACKGROUND

Hydrocarbons, and specifically olefins such as ethylene, are typically building blocks used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Ethylene can also be produced by oxidative coupling of the methane (OCM) as represented by Equations (I) and (II):

2CH₄+O₂→C₂H₄+2H₂O ΔH=−67 kcal/mol (I)

2CH₄+½O₂→C₂H₄+H₂O ΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂O ΔH=−124 kcal/mol (III)

CH₄+2O₂→CO₂+2H₂O ΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C-H bond breakage. The endothermic nature of the bond breakage is due to the chemical stability of methane, which is a chemically stable molecule due to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Furthermore, the produced ethylene is highly reactive and can form unwanted and thermodynamically favored oxidation products.

There have been attempts to control the exothermic reaction of the OCM by using alternating layers of selective OCM catalysts; through the use of fluidized bed reactors; and/or by using steam as a diluent. However, these solutions are costly and inefficient. For example, a large amount of water (steam) is required to absorb the heat of the reaction. Thus, there is an ongoing need for the development of OCM processes.

### BRIEF SUMMARY

Disclosed herein is a method for producing olefins comprising (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 1,000° C., and wherein the reactor is characterized by a residence time of from about 1 millisecond to about 100 milliseconds in the catalyst bed, (b) wherein isothermal reactor conditions minimize hot spots formation in the catalyst bed, thereby decreasing an incidence of deep oxidation reactions, when compared to an incidence of deep oxidation reactions in an otherwise similar oxidative coupling of CH₄ reaction conducted under non-isothermal conditions, (c) allowing at least a portion of the reactant mixture to contact the catalyst and react via an oxidative coupling of CH₄ reaction to form a product mixture under isothermal conditions, wherein the product mixture comprises C₂₊ hydrocarbons and synthesis gas, wherein the C₂₊ hydrocarbons comprise olefins and paraffins, wherein the C₂₊ hydrocarbons comprise C₂ hydrocarbons and C₃ hydrocarbons, wherein the product mixture is characterized by an olefin/paraffin molar ratio of from about 0.5:1 to about 20:1, wherein the synthesis gas comprises hydrogen (H₂) and carbon monoxide (CO), and wherein the product mixture is characterized by a H₂/CO molar ratio of from about 0.2:1 to about 2.5:1, (d) recovering at least a portion of the product mixture from the reactor, and (e) recovering at least a portion of the C₂ hydrocarbons and/or at least a portion of the synthesis gas from the product mixture.

Also disclosed herein is a method for producing olefins comprising (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 950° C., and wherein the reactor is characterized by a residence time of from about 1 millisecond to about 100 milliseconds in the catalyst bed, and wherein isothermal reactor conditions minimize hot spots formation within the reactor, (b) allowing at least a portion of the reactant mixture to contact the catalyst and react via an oxidative coupling of CH₄ reaction to form a product mixture, wherein the product mixture comprises olefins, and wherein a selectivity to olefins is increased by equal to or greater than about 10% when compared to a selectivity of an otherwise similar oxidative coupling of CH₄ reaction conducted under non-isothermal conditions, and (c) recovering at least a portion of the product mixture from the reactor.

Further disclosed herein is a method for producing ethylene comprising (a) introducing a reactant mixture to an isothermal reactor, wherein the reactant mixture comprises methane (CH₄) and oxygen (O₂), wherein the reactant mixture is characterized by a CH₄/O₂ molar ratio of from about 4:1 to about 8:1, wherein the isothermal reactor comprises a catalyst bed comprising a catalyst, wherein an isothermal reaction temperature in the catalyst bed is from about 800° C. to about 900° C., and wherein the reactor is characterized by a residence time of from about 10 millisecond to about 50 milliseconds in the catalyst bed, (b) allowing at least a portion of the reactant mixture to contact the catalyst and react via an oxidative coupling of CH₄ reaction to form a product mixture, wherein the product mixture comprises ethylene, and wherein a selectivity to ethylene is increased by equal to or greater than about 40% when compared to a selectivity of an otherwise similar oxidative coupling of CH₄ reaction conducted under non-isothermal conditions, (c) recovering at least a portion of the product mixture from the reactor, and (d) separating at least a portion of the ethylene from the product mixture by cryogenic distillation to yield recovered ethylene.

### DETAILED DESCRIPTION

Disclosed herein are methods for producing olefins comprising (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 1,000° C., and wherein the reactor is characterized by a residence time of from about 1 millisecond to about 100 milliseconds in the catalyst bed; (b) wherein isothermal reactor conditions minimize hot spots formation in the catalyst bed, thereby decreasing an incidence of deep oxidation reactions, when compared to an incidence of deep oxidation reactions in an otherwise similar oxidative coupling of CH₄ reaction conducted under non-isothermal conditions; (c) allowing at least a portion of the reactant mixture to contact the catalyst and react via an oxidative coupling of CH₄ reaction to form a product mixture under isothermal conditions, wherein the product mixture comprises C₂₊ hydrocarbons and synthesis gas, wherein the C₂₊ hydrocarbons comprise olefins and paraffins, wherein the C₂₊ hydrocarbons comprise C₂ hydrocarbons and C₃ hydrocarbons, wherein the product mixture is characterized by an olefin/paraffin molar ratio of from about 0.5:1 to about 20:1, wherein the synthesis gas comprises hydrogen (H₂) and carbon monoxide (CO), and wherein the product mixture is characterized by a H₂/CO molar ratio of from about 0.2:1 to about 2.5:1; (d) recovering at least a portion of the product mixture from the reactor; and (e) recovering at least a portion of the C₂ hydrocarbons and/or at least a portion of the synthesis gas from the product mixture. In an embodiment, the method for producing olefins can further comprise minimizing deep oxidation of methane to carbon dioxide (CO₂), wherein the product mixture comprises less than about 10 mol % CO₂.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an embodiment,” “another embodiment,” “other embodiments,” “some embodiments,” and so forth, means that a particular element (e.g., feature, structure, property, and/or characteristic) described in connection with the embodiment is included in at least an embodiment described herein, and may or may not be present in other embodiments. In addition, it is to be understood that the described element(s) can be combined in any suitable manner in the various embodiments.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“—”) that is not between two letters or symbols is used to indicate a point of attachment for a substituent. For example, —CHO is attached through the carbon of the carbonyl group.

In an embodiment, a method for producing olefins can comprise introducing a reactant mixture to an isothermal reactor, wherein the reactant mixture comprises methane (CH₄) and oxygen (O₂); and allowing at least a portion of the reactant mixture to contact a catalyst and react via an oxidative coupling of CH₄ (OCM) reaction to form a product mixture under isothermal conditions.

The OCM has been the target of intense scientific and commercial interest for more than thirty years due to the tremendous potential of such technology to reduce costs, energy, and environmental emissions in the production of ethylene (C₂H₄). As an overall reaction, in the OCM, CH₄ and O₂ react exothermically over a catalyst to form C₂H₄, water (H₂O) and heat.

Generally, in the OCM, CH₄ is first oxidatively converted into ethane (C₂H₆), and then into C₂H₄. CH₄ is activated heterogeneously on a catalyst surface, forming methyl free radicals (e.g., CH₃.), which then couple in a gas phase to form C₂H₆. C₂H₆ subsequently undergoes dehydrogenation to form C₂H₄. An overall yield of desired C₂ hydrocarbons is reduced by non-selective reactions of methyl radicals with the catalyst surface and/or oxygen in the gas phase, which produce (undesirable) carbon monoxide and carbon dioxide. Some of the best reported OCM outcomes encompass a ˜20% conversion of methane and ˜80% selectivity to desired C₂ hydrocarbons.

In an embodiment, the reactant mixture can comprise a hydrocarbon or mixtures of hydrocarbons, and oxygen. In some embodiments, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons such as diesel fuel, jet fuel, gasoline, tars, kerosene, etc.), oxygenated hydrocarbons, biodiesel, alcohols, dimethyl ether, and the like, or combinations thereof. In an embodiment, the reactant mixture can comprise CH₄ and O₂.

In an embodiment, the O₂ used in the reaction mixture can be oxygen gas (which may be obtained via a membrane separation process), technical oxygen (which may contain some air), air, oxygen enriched air, and the like, or combinations thereof.

In an embodiment, the reactant mixture can be a gaseous mixture. In an embodiment, the reactant mixture can be characterized by a CH₄/O₂ molar ratio of from about 2:1 to about 40:1, alternatively from about 3:1 to about 25:1, alternatively from about 3:1 to about 16:1, alternatively from about 4:1 to about 12:1, or alternatively from about 4:1 to about 8:1.

In an embodiment, the reactant mixture can further comprise a diluent. The diluent is inert with respect to the OCM reaction, e.g., the diluent does not participate in the OCM reaction.

In an embodiment, the diluent can comprise water, nitrogen, inert gases, and the like, or combinations thereof. In an embodiment, the diluent contributes to isothermal conditions of reactor, as will be described in more detail later herein.

In an embodiment, the diluent can be present in the reactant mixture in an amount of from about 0.5% to about 80%, alternatively from about 5% to about 50%, or alternatively from about 10% to about 30%, based on the total volume of the reactant mixture.

In an embodiment, a method for producing olefins can comprise introducing the reactant mixture to an isothermal reactor, wherein the reactor comprises a catalyst. Generally, an isothermal reactor refers to a reactor that has the ability of maintaining a substantially constant reaction temperature (e.g., isothermal conditions, isothermal reaction conditions, isothermal reactor conditions, etc.), through a heat exchange system such as a heat exchange jacket. For purposes of the disclosure herein, a substantially constant temperature can be defined as a temperature that varies by less than about +10° C., alternatively less than about ±9° C., alternatively less than about ±8° C., alternatively less than about ±7° C., alternatively less than about ±6° C., alternatively less than about ±5° C., alternatively less than about ±4° C., alternatively less than about ±3° C., alternatively less than about ±2° C., or alternatively less than about ±1° C.

In an embodiment, isothermal reactor conditions can minimize hot spots formation within the reactor (e.g., hot spots formation in the catalyst bed). Generally, hot spots are portions (e.g., areas) of catalyst that exceed the reaction temperature, and such hot spots can lead to thermal deactivation of the catalyst and/or enhancement of deep oxidation reactions. Deep oxidation reactions include oxidation of methane to CO_{y} (e.g., CO, CO₂).

In an embodiment, the isothermal reactor can comprise a fixed bed reactor, wherein the fixed bed comprises catalyst bed. In an embodiment, the isothermal reactor can comprise a tubular reactor, a cooled tubular reactor, a continuous flow reactor, and the like, or combinations thereof.

In an embodiment, the isothermal reactor can comprise a reactor vessel located inside a fluidized sand bath reactor, wherein the fluidized sand bath provides isothermal conditions (i.e., substantially constant temperature) for the reactor. In such embodiment, the fluidized sand bath reactor can be a fixed bed reactor comprising an outer jacket comprising a fluidized sand bath. The fluidized sand bath can exchange heat with the reactor, thereby providing isothermal conditions for the reactor. Generally, a fluidized bath employs fluidization of a mass of finely divided inert particles (e.g., sand particles, metal oxide particles, aluminum oxide particles, metal oxides microspheres, quartz sand microspheres, aluminum oxide microspheres, silicon carbide microspheres) by means of an upward stream of gas, such as for example air, nitrogen, etc.

In an embodiment, the isothermal conditions can be provided by fluidization of heated microspheres around the isothermal reactor comprising the catalyst bed, wherein the microspheres can be heated at a temperature of from about 725° C. to about 1,000° C., alternatively from about 750° C. to about 950° C., or alternatively from about 800° C. to about 900° C.; and wherein the microspheres can comprise sand, metal oxides, quartz sand, aluminum oxide, silicon carbide, and the like, or combinations thereof. In an embodiment, the microspheres (e.g., inert particles) can have a size of from about 10 mesh to about 400 mesh, alternatively from about 30 mesh to about 200 mesh, or alternatively from about 80 mesh to about 100 mesh, based on U.S. Standard Sieve Size.

While in a fluidized state, the individual inert particles become microscopically separated from each other by the upward moving stream of gas. Generally, a fluidized bath behaves remarkably like a liquid, exhibiting characteristics which are generally attributable to a liquid state (e.g., a fluidized bed can be agitated and bubbled; inert particles of less density will float while those with densities greater than the equivalent fluidized bed density will sink; heat transfer characteristics between the fluidized bed and a solid interface can have an efficiency approaching that of an agitated liquid; etc.).

In an embodiment, isothermal conditions can be provided by fluidized aluminum oxide, such as for example by a BFS high temperature furnace, which is a high temperature calibration bath, and which is commercially available from Techne Calibration.

In an embodiment, the reaction mixture can be introduced to the isothermal reactor at a temperature of from about 150° C. to about 300° C., alternatively from about 175° C. to about 250° C., or alternatively from about 200° C. to about 225° C. As will be appreciated by one of skill in the art, and with the help of this disclosure, while the OCM reaction is exothermic, heat input is necessary for promoting the formation of methyl radicals from CH₄, as the C—H bonds of CH₄ are very stable, and the formation of methyl radicals from CH₄ is endothermic. In an embodiment, the reaction mixture can be introduced to the isothermal reactor at a temperature effective to promote an OCM reaction.

In an embodiment, the isothermal reactor can be characterized by a temperature (e.g., an isothermal reaction temperature in a catalyst bed) of less than about 1,000° C., alternatively less than about 950° C., or alternatively less than about 900° C. In an embodiment, an isothermal reaction temperature in the catalyst bed can be from about 750° C. to about 1,000° C., alternatively from about 750° C. to about 950° C., or alternatively from about 800° C. to about 950° C., wherein the catalyst bed comprises a catalyst. As will be appreciated by one of skill in the art, and with the help of this disclosure, different catalysts have different deactivation temperatures (Td), and as such the reactor temperature (e.g., an isothermal reaction temperature in a catalyst bed) can vary based on the type of catalyst used.

In an embodiment, the diluent can contribute to the isothermal conditions of the reactor. In some embodiments, the diluent can physically interact with the catalyst (e.g., a portion of the diluent can be adsorbed on the catalyst surface) thereby decreasing catalyst activity. Without wishing to be limited by theory, when the diluent is adsorbed onto the catalyst surface, fewer catalyst active sites are available for the OCM, and consequently the overall rate of the OCM is slower (as opposed to no diluent adsorbed onto the catalyst surface), thereby allowing more time for removing the heat produced by the exothermic OCM reaction.

In an embodiment, the diluent can provide for heat control of the OCM reaction, e.g., the diluent can act as a heat sink. Generally, an inert compound (e.g., a diluent) can absorb some of the heat produced in the exothermic OCM reaction, without degrading or participating in any reaction (OCM or other reaction), thereby providing for controlling a temperature inside the reactor. As will be appreciated by one of skill in the art, and with the help of his disclosure, the diluent can be introduced to the reactor at ambient temperature, or as part of the reaction mixture (at a reaction mixture temperature), and as such the temperature of the diluent entering the rector is much lower than the reaction temperature, and the diluent can act as a heat sink.

In an embodiment, the isothermal reactor can be characterized by a pressure of from about ambient pressure (e.g., atmospheric pressure) to about 500 psig, alternatively from about ambient pressure to about 200 psig, or alternatively from about ambient pressure to about 100 psig. In an embodiment, the method for producing olefins as disclosed herein can be carried out at ambient pressure.

In an embodiment, the isothermal reactor can be characterized by a residence time in a catalyst bed of from about 1 millisecond (ms) to about 100 ms, alternatively from about 10 ms to about 50 ms, or alternatively from about 15 ms to about 25 ms, wherein the catalyst bed comprises a catalyst. Generally, the residence time of a reactor refers to the average amount of time that a compound (e.g., a molecule of that compound) spends in that particular reactor, and specifically, the residence time in a catalyst bed refers to the average amount of time that a compound (e.g., a molecule of that compound) spends in that particular catalyst bed, e.g., the average amount of time that it takes for a compound (e.g., a molecule of that compound) to travel through the catalyst bed.

In an embodiment, the isothermal reactor can be characterized by a weight hourly space velocity of from about 3,600 h⁻¹ to about 36,000 ⁻¹, alternatively from about 5,000 h⁻¹ to about 35,000 ⁻¹, or alternatively from about 10,000 h⁻¹ to about 30,000 ⁻¹. Generally, the weight hourly space velocity refers to a mass of reagents fed per hour divided by a mass of catalyst used in a particular reactor.

In an embodiment, the isothermal reactor can comprise a catalyst bed comprising a catalyst, wherein the catalyst catalyzes the OCM reaction (e.g., the catalyst catalyzes a high temperature oxidative coupling or conversion of CH₄ to C₂ hydrocarbons and synthesis gas). In such embodiment, the catalyst can comprise basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; and the like; or combinations thereof.

In an embodiment, the catalysts suitable for use in the present disclosure can be supported catalysts and/or unsupported catalysts. In some embodiments, the supported catalyst can comprise a support, wherein the support can be catalytically active (e.g., the support can catalyze an OCM reaction). In other embodiments, the supported catalyst can comprise a support, wherein the support can be catalytically inactive (e.g., the support cannot catalyze an OCM reaction). In yet other embodiments, the supported catalyst can comprise a catalytically active support and a catalytically inactive support. Nonlimiting examples of a catalyst support suitable for use in the present disclosure include MgO, Al₂O₃, SiO₂, and the like, or combinations thereof. As will be appreciated by one of skill in the art, and with the help of this disclosure, the support can be purchased or can be prepared by using any suitable methodology, such as for example precipitation/co-precipitation, sol-gel techniques, templates/surface derivatized metal oxides synthesis, solid-state synthesis of mixed metal oxides, microemulsion techniques, solvothermal techniques, sonochemical techniques, combustion synthesis, etc.

In an embodiment, the catalyst can comprise one or more metals (e.g., catalytic metals), one or more metal compounds (e.g., compounds of catalytic metals), and the like, or combinations thereof. Nonlimiting examples of catalytic metals suitable for use in the present disclosure include Li, Na, Ca, Cs, Mg, La, Ce, W, Mn, and the like, or combinations thereof. Nonlimiting examples of catalysts suitable for use in the present disclosure include La on a MgO support, Na, Mn, and La₂O₃ on an alumina support, Na and Mn oxides on a silicon dioxide support, Na₂WO₄ and Mn on a silicon dioxide support, and the like, or combinations thereof.

In an embodiment, a catalyst that can promote an OCM reaction to produce ethylene can comprise Li₂O, Na₂O, Cs₂O, MgO, WO₃, Mn₃O₄, and the like, or combinations thereof. In some embodiments, the catalyst can comprise a catalyst mixture, such as for example a catalyst mixture comprising a first supported catalyst comprising Ce and La, and a second supported catalyst comprising Mn, W, and Na.

Nonlimiting examples of catalysts suitable for use in the present disclosure include CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Sr—La/CeO₂, Sr—Ce/La₂O₃, Na—Mn—La₂O₃/Al₂O₃, Na—Mn—O/SiO₂, Na₂WO₄—Mn/SiO₂, Na₂WO₄—Mn—O/SiO₂, and the like, or combinations thereof.

In an embodiment, the catalyst can be characterized by a deactivation temperature (Td). Generally, the Td of a catalyst represents the temperature at which the catalyst loses catalytic ability (e.g., loses the ability to catalyze the OCM reaction) due to thermal degradation of the catalyst. Thermal degradation of a catalyst can involve a variety of distinct processes, such as coking (e.g., agglomeration of material such as carbon deposits on a catalyst surface); sintering of catalytically active sites (e.g., agglomeration of catalytically active sites with a reduction in catalytically active surface area); evaporation of promoters from the catalyst and the like; or combinations thereof. In an embodiment, loss of catalytic activity can be related to a loss of methane and/or oxygen conversion, wherein oxygen conversion can be reduced by from about 100% to about 95%, alternatively from about 99.9% to about 98.0%, or alternatively from about 99.9% to 99.5%, within 500 hours of catalyst use. In such embodiment, the loss of catalytic activity can be due to a loss of some components from the catalyst, fusing of active material to a non-active catalyst phase, and the like, or combinations thereof.

In an embodiment, the catalyst can be characterized by a Td of equal to or greater than about 950° C., alternatively equal to or greater than about 900° C., or alternatively equal to or greater than about 800° C.

In an embodiment, a method for producing olefins can comprise allowing at least a portion of the reactant mixture to contact the catalyst and react via an oxidative coupling of CH₄ reaction to form a product mixture, wherein the product mixture comprises olefins, and wherein a selectivity to olefins is increased by equal to or greater than about 10%, alternatively equal to or greater than about 20%, or alternatively equal to or greater than about 30%, when compared to a selectivity of an otherwise similar oxidative coupling of CH₄ reaction conducted under non-isothermal conditions.

Generally, a selectivity to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a Cₓ selectivity (e.g., C₂ selectivity, C₂₊ selectivity, C_{olefins} selectivity, etc.) can be calculated by dividing a number of moles of carbon (C) from CH₄ that were converted into the desired product (e.g., C_{C2H4}, C_{c2H6}, C_{olefins}, etc.) by the total number of moles of C from CH₄ that were converted (e.g., C_{C2H4}, C_{C2H6}, C_{C2H2}, C_{C3H6}, C_{C3H8}, C_{C4s}, C_{CO2}, C_{CO}, etc.). C_{C2H4}=number of moles of C from CH₄ that were converted into C₂H₄; C_{C2H6}=number of moles of C from CH₄ that were converted into C₂H₆; C_{C2H2}=number of moles of C from CH₄ that were converted into C₂H₂; C_{C3H6}=number of moles of C from CH₄ that were converted into C₃H₆; C_{C3H8}=number of moles of C from CH₄ that were converted into C₃H₈; C_{C4s}=number of moles of C from CH₄ that were converted into C₄ hydrocarbons (C₄s); C_{CO2}=number of moles of C from CH₄ that were converted into CO₂; C_{CO}=number of moles of C from CH₄ that were converted into CO; C_{olefins}=number of moles of C from CH₄ that were converted into olefins (e.g., C₂H₄, C₃H₆, etc.); etc.

In an embodiment, the product mixture comprises coupling products, partial oxidation products (e.g., partial conversion products, such as CO, H₂, CO₂), and unreacted methane. In an embodiment, the coupling products can comprise olefins (e.g., alkenes, characterized by a general formula CₙH₂ₙ) and paraffins (e.g., alkanes, characterized by a general formula CₙH₂ₙ₊₂).

In an embodiment, the product mixture can comprise olefins and paraffins. In such embodiment, a molar ratio of olefins to paraffins can be from about 0.5:1 to about 20:1, alternatively from about 1:1 to about 20:1, alternatively from about 1:1 to about 10:1, or alternatively from about 1:1 to about 5:1. In an embodiment, an olefin/paraffin molar ratio in the product mixture can be higher than an olefin/paraffin molar ratio in a product mixture produced by an otherwise similar OCM reaction conducted under non-isothermal conditions. In some embodiments, an olefin content of the product mixture can be higher than a paraffin content of the product mixture.

In an embodiment, the product mixture can comprise C₂₊ hydrocarbons and synthesis gas, wherein the C₂₊ hydrocarbons can comprise C₂ hydrocarbons and C₃ hydrocarbons. In an embodiment, the C₂₊ hydrocarbons can further comprise C₄ hydrocarbons (C₄s), such as for example butane, iso-butane, n-butane, butylene, etc. In some embodiments, the product mixture can comprise C₂H₄, C₂H₆, CH₄, CO, H₂, CO₂ and H₂O.

In an embodiment, the C₂ hydrocarbons can comprise C₂H₄ and C₂H₆. In such embodiment, a molar ratio of C₂H₄ to C₂H₆ can be from about 0.5:1 to about 20:1, alternatively from about 1:1 to about 20:1, alternatively from about 1:1 to about 10:1, or alternatively from about 1:1 to about 5:1. In an embodiment, a C₂H₄/C₂H₆ molar ratio in the product mixture can be higher than a C₂H₄/C₂H₆ molar ratio in a product mixture produced by an otherwise similar OCM reaction conducted under non-isothermal conditions. In some embodiments, a C₂H₄ content of the product mixture can be higher than a C₂H₆ content of the product mixture. In an embodiment, the C₂ hydrocarbons can further comprise acetylene (C₂H₂).

In an embodiment, the C₃ hydrocarbons can comprise propylene (C₃H₆) and propane (C₃H₈). In such embodiment, a molar ratio of C₃H₆ to C₃H₈ can be from about 0.5:1 to about 50:1, alternatively from about 1:1 to about 25:1, or alternatively from about 2:1 to about 20:1.

In an embodiment, a selectivity to C₂₊ hydrocarbons and synthesis gas (e.g., C_{2+&SG} selectivity) can be from about 60% to about 99%, alternatively from about 70% to about 98%, alternatively from about 75% to about 97%, or alternatively from about 80% to about 95%. The C_{2+&SG} selectivity refers to how much C₂₊ hydrocarbons and synthesis gas (e.g., desired products, such as C₂ hydrocarbons, C₃ hydrocarbons, C₄s, CO for synthesis gas, etc.) were formed divided by the total products formed, including C₂H₄, C₃H₆, C₂H₆, C₃H₈, C₂H₂, C₄s, CO₂ and CO. For example, the C_{2+&SG} selectivity can be calculated by using equation (1):

$${C}_{2 + \& SG} selectivity = \frac{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{CO}\end{matrix}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4} s} + {C}_{{CO}_{2}} + {C}_{CO}\end{matrix}} 100 \% \qquad ( 1 )$$

As will be appreciated by one of skill in the art, if a specific product and/or hydrocarbon product is not produced in a certain OCM reaction/process, then the corresponding C_{Cx} is 0, and the term is simply removed from selectivity calculations.

In an embodiment, a selectivity to olefins (e.g., C_{olefins} selectivity) can be from about 50% to about 80%, alternatively from about 55% to about 75%, or alternatively from about 60% to about 70%. The C_{olefins} selectivity refers to how much C₂H₄ and C₃H₆ were formed divided by the total products formed, including C₂H₄, C₃H₆, C₂H₆, C₃H₈, C₂H₂, C₄s, CO₂ and CO. For example, the C_{olefins} selectivity can be calculated by using equation (2):

$${C}_{olefins} selectivity = \frac{2 {C}_{{C}_{2} {H}_{4}} + 3 {C}_{{C}_{3} {H}_{6}}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4} s} + {C}_{{CO}_{2}} + {C}_{CO}\end{matrix}} 100 \% \qquad ( 2 )$$

In an embodiment, a selectivity to ethylene (C₂₌ selectivity) can be from about 20% to about 80%, alternatively from about 30% to about 75%, alternatively from about 40% to about 70%, or alternatively from about 50% to about 65%. The C₂₋ selectivity refers to how much C₂H₄ was formed divided by the total products formed, including C₂H₄, C₃H₆, C₂H₆, C₃H₈, C₂H₂, C₄s, CO₂ and CO. For example, the selectivity to ethylene can be calculated by using equation (3):

$${C}_{2 =} selectivity = \frac{2 {C}_{{C}_{2} {H}_{4}}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4} s} + {C}_{{CO}_{2}} + {C}_{CO}\end{matrix}} 100 \% \qquad ( 3 )$$

In an embodiment, a selectivity to C₂ hydrocarbons (C₂ selectivity) can be from about 55% to about 95%, alternatively from about 60% to about 90%, or alternatively from about 65% to about 85%. The C₂ selectivity refers to how much C₂H₄, C₂H₆, and C₂H₂ were formed divided by the total products formed, including C₂H₄, C₃H₆, C₂H₆, C₃H₈, C₂H₂, C₄s, CO₂ and CO. For example, the C₂ selectivity can be calculated by using equation (4):

$${C}_{2} selectivity = \frac{2 {C}_{{C}_{2} {H}_{4}} + 2 {C}_{{C}_{2} {H}_{6}} + 2 {C}_{{C}_{2} {H}_{2}}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4} s} + {C}_{{CO}_{2}} + {C}_{CO}\end{matrix}} 100 \% \qquad ( 4 )$$

In an embodiment, a selectivity to C₂₋ hydrocarbons (C₂₊ selectivity) can be from about 60% to about 95%, alternatively from about 65% to about 90%, or alternatively from about 70% to about 85%. The C₂₊ selectivity refers to how much C₂H₄, C₂H₆, C₂H₂, C₃H₆, C₃H₈, and C₄s were formed divided by the total products formed, including C₂H₄, C₃H₆, C₂H₆, C₃H₈, C₂H₂, C₄s, CO₂ and CO. For example, the C₂₊ selectivity can be calculated by using equation (5):

$${C}_{2 +} selectivity = \frac{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s}\end{matrix}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4} s} + {C}_{{CO}_{2}} + {C}_{CO}\end{matrix}} 100 \% \qquad ( 5 )$$

In some embodiments, the C₂₊ hydrocarbons can further comprise C₄ hydrocarbons (e.g., butane, butylene, etc.). As will be appreciated by one of skill in the art, and with the help of this disclosure, if any C₄ hydrocarbons are formed during the OCM, the amount of C₄ hydrocarbons formed is low enough such that it would not affect the calculation of C₂₊ selectivity.

In an embodiment, a methane conversion can be from about 10% to about 45%, alternatively from about 12.5% to about 40%, alternatively from about 15% to about 35%, or alternatively from about 20% to about 30%. Generally, a conversion of a reagent or reactant refers to the percentage (usually mol %) of reagent that reacted to both undesired and desired products, based on the total amount (e.g., moles) of reagent present before any reaction took place. For purposes of the disclosure herein, the conversion of a reagent is a % conversion based on moles converted. For example, the methane conversion can be calculated by using equation (6):

$${CH}_{4} conversion = \frac{{C}_{{CH}_{4}}^{in} - {C}_{{CH}_{4}}^{out}}{{C}_{{CH}_{4}}^{in}} 100 \% \qquad ( 6 )$$

wherein C_{CH}_{₄}ⁱⁿ=number of moles of C from CH₄ that entered the reactor as part of the reactant mixture; and C_{CH}_{₄}^{out}=number of moles of C from CH₄ that was recovered from the reactor as part of the product ₄ mixture.

In an embodiment, a sum of CH₄ conversion plus the selectivity to C₂₊ hydrocarbons can be equal to or greater than about 100%, alternatively equal to or greater than about 105%, or alternatively equal to or greater than about 110%. As will be appreciated by one of skill in the art, and with the help of this disclosure, the lower the residence time, the higher the selectivity to desired products, and the lower the methane conversion. Further, as will be appreciated by one of skill in the art, and with the help of this disclosure, the higher the reaction temperature, the higher the selectivity to desired products (e.g., olefins, hydrocarbons, etc.); however, generally, extremely high reaction temperatures (e.g., over about 1,000° C.) can lead to an increase in deep oxidation products (e.g., CO, CO₂).

In an embodiment, a method for producing olefins can further comprise minimizing deep oxidation of methane to CO₂. In an embodiment, the product mixture can comprise less than about 10 mol % CO₂, alternatively less than about 7.5 mol % CO₂, or alternatively less than about 5 mol % CO₂.

In an embodiment, equal to or greater than about 5 mol %, alternatively equal to or greater than about 10 mol %, or alternatively equal to or greater than about 15 mol % of the reactant mixture can be converted to olefins.

In an embodiment, equal to or greater than about 5 mol %, alternatively equal to or greater than about 10 mol %, or alternatively equal to or greater than about 15 mol % of the reactant mixture can be converted to ethylene.

In an embodiment, equal to or greater than about 10 mol %, alternatively equal to or greater than about 15 mol %, or alternatively equal to or greater than about 20 mol % of the reactant mixture can be converted to C₂ hydrocarbons.

In an embodiment, equal to or greater than about 12 mol %, alternatively equal to or greater than about 17 mol %, or alternatively equal to or greater than about 22 mol % of the reactant mixture can be converted to C₂₊ hydrocarbons.

In an embodiment, equal to or greater than about 5 mol %, alternatively equal to or greater than about 10 mol %, or alternatively equal to or greater than about 15 mol % of the reactant mixture can be converted to synthesis gas. Generally, in industrial settings, synthesis gas is produced by an endothermic process of steam reforming of natural gas. In an embodiment, the synthesis gas can be produced as disclosed herein as a side reaction in an OCM reaction/process.

In an embodiment, the product mixture can comprise synthesis gas (e.g., CO and H₂). Synthesis gas, also known as syngas, is generally a gas mixture consisting primarily of CO and H₂, and sometimes CO₂. Synthesis gas can be used for producing olefins; for producing methanol; for producing ammonia and fertilizers; in the steel industry; as a fuel source (e.g., for electricity generation); etc. In such embodiment, the product mixture (e.g., the synthesis gas of the product mixture) can be characterized by a hydrogen (H₂) to carbon monoxide (CO) ratio of from about 0.2:1 to about 2.5:1, alternatively from about 0.5:1 to about 2.5:1, alternatively from about 0.2:1 to about 1.8:1, alternatively from about 1:1 to about 2.25:1, alternatively from about 1.3:1 to about 2.2:1, or alternatively from about 1.5:1 to about 2:1.

In an embodiment, a selectivity to CO (C_{CO} selectivity) can be from about 5% to about 25%, alternatively from about 7.5% to about 22.5%, or alternatively from about 10% to about 20%. The C_{CO} selectivity refers to how much CO was formed divided by the total products formed, including C₂H₄, C₃H₆, C₂H₆, C₃H₈, C₂H₂, C₄s, CO₂ and CO. For example, the C_{CO} selectivity can be calculated by using equation (7):

$${C}_{CO} selectivity = \frac{{C}_{CO}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4} s} + {C}_{{CO}_{2}} + {C}_{CO}\end{matrix}} 100 \% \qquad ( 7 )$$

In an embodiment, at least a portion of the synthesis gas can be separated from the product mixture to yield recovered synthesis gas, for example by cryogenic distillation. As will be appreciated by one of skill in the art, and with the help of this disclosure, the recovery of synthesis gas is done as a simultaneous recovery of both H₂ and CO.

In an embodiment, at least a portion of the recovered synthesis gas can be further converted to olefins. For example, the recovered synthesis gas can be converted to alkanes by using a Fisher-Tropsch process, and the alkanes can be further converted by dehydrogenation into olefins.

In an embodiment, at least a portion of the unreacted methane and at least a portion of the synthesis gas can be separated from the product mixture to yield a recovered synthesis gas mixture, wherein the recovered synthesis gas mixture comprises CO, H₂, and CH₄. In an embodiment, at least a portion of the recovered synthesis gas mixture can be further converted to olefins. In some embodiments, at least a portion of the recovered synthesis gas mixture can be further used as fuel to generate power. In other embodiments, at least a portion of the unreacted methane can be recovered and recycled to the reactant mixture.

In an embodiment, at least a portion of the recovered synthesis gas mixture can be further converted to liquid hydrocarbons (e.g., alkanes) by a Fisher-Tropsch process. In such embodiment, the liquid hydrocarbons can be further converted by dehydrogenation into olefins.

In some embodiments, at least a portion of the recovered synthesis gas mixture can be further converted to methane via a methanation process.

In an embodiment, a method for producing olefins can comprise recovering at least a portion of the product mixture from the reactor, wherein the product mixture can be collected as an outlet gas mixture from the reactor. In an embodiment, a method for producing olefins can comprise recovering at least a portion of the C₂ hydrocarbons and/or at least a portion of the synthesis gas from the product mixture. In an embodiment, the product mixture can comprise C₂₊ hydrocarbons (including olefins), unreacted methane, and optionally a diluent. When water (e.g., steam) is used as a diluent, the water can be separated from the product mixture prior to separating any of the other product mixture components. For example, by cooling down the product mixture to a temperature where the water condenses (e.g., below 100° C. at ambient pressure), the water can be removed from the product mixture, by using a flash chamber for example.

In an embodiment, at least a portion of the C₂₊ hydrocarbons can be separated (e.g. recovered) from the product mixture to yield recovered C₂₊ hydrocarbons. The C₂₊ hydrocarbons can be separated from the product mixture by using any suitable separation technique. In an embodiment, at least a portion of the C₂₊ hydrocarbons can be separated from the product mixture by distillation (e.g., cryogenic distillation).

In an embodiment, at least a portion of the recovered C₂₊ hydrocarbons can be used for ethylene production. In some embodiments, at least a portion of ethylene can be separated from the product mixture (e.g., from the C₂₊ hydrocarbons, from the recovered C₂₊ hydrocarbons) to yield recovered ethylene and recovered hydrocarbons, by using any suitable separation technique (e.g., distillation). In other embodiments, at least a portion of the recovered hydrocarbons (e.g., recovered C₂₊ hydrocarbons after olefin separation, such as separation of C₂H₄ and C₃H₆) can be converted to ethylene, for example by a conventional steam cracking process.

In an embodiment, at least a portion of the unreacted methane can be separated from the product mixture to yield recovered methane. Methane can be separated from the product mixture by using any suitable separation technique, such as for example distillation (e.g., cryogenic distillation). In an embodiment, at least a portion of the recovered methane can be recycled to the reactant mixture.

In an embodiment, a method for producing olefins can comprise (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 1,000° C., and wherein the reactor is characterized by a residence time of from about 1 millisecond to about 100 milliseconds in the catalyst bed; (b) wherein isothermal reactor conditions minimize hot spots formation in the catalyst bed, thereby decreasing an incidence of deep oxidation reactions, when compared to an incidence of deep oxidation reactions in an otherwise similar oxidative coupling of CH₄ reaction conducted under non-isothermal conditions; (c) allowing at least a portion of the reactant mixture to contact the catalyst and react via an oxidative coupling of CH₄ reaction to form a product mixture, wherein the product mixture comprises C₂₊ hydrocarbons (e.g., olefins, paraffins) and synthesis gas (syngas) (e.g., the product mixture can comprise C₂H₄, C₂H₆, CH₄, CO, H₂, CO₂, H₂O, etc.), wherein an olefin/paraffin molar ratio in the product mixture is higher than an olefin/paraffin molar ratio in a product mixture produced by an otherwise similar OCM reaction conducted under non-isothermal conditions, wherein a C₂H₄/C₂H₆ molar ratio is greater than 1:1, and wherein a selectivity to ethylene is increased by equal to or greater than about 10% when compared to a C₂₌ selectivity of an otherwise similar OCM reaction conducted under non-isothermal conditions; and (d) recovering at least a portion of the product mixture from the reactor, wherein the product mixture is collected as an outlet gas mixture from the reactor.

In an embodiment, a method for producing olefins can comprise (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 950° C., and wherein the reactor is characterized by a residence time of from about 1 millisecond to about 100 milliseconds in the catalyst bed; (b) wherein isothermal reactor conditions minimize hot spots formation in the catalyst bed, thereby decreasing an incidence of deep oxidation reactions, when compared to an incidence of deep oxidation reactions in an otherwise similar oxidative coupling of CH₄ reaction conducted under non-isothermal conditions; (c) allowing at least a portion of the reactant mixture to contact the catalyst and react via an oxidative coupling of CH₄ reaction to form a product mixture, wherein the product mixture comprises C₂₊ hydrocarbons (e.g., olefins, such as ethylene; paraffins, such as ethane) and partial conversion products (e.g., CO, H₂, CO₂) such as synthesis gas (syngas) (e.g., the product mixture can comprise C₂H₄, C₂H₆, CH₄, CO, H₂, CO₂, H₂O, etc.), wherein an olefin/paraffin molar ratio in the product mixture is higher than an olefin/paraffin molar ratio in a product mixture produced by an otherwise similar OCM reaction conducted under non-isothermal conditions, wherein an olefin/paraffin molar ratio in the product mixture can be from about 0.5:1 to about 20:1, and wherein a H₂/CO molar ratio in the product mixture can be from about 0.2:1 to about 1.8:1; (d) recovering at least a portion of the product mixture from the reactor, wherein the product mixture is collected as an outlet gas mixture from the reactor; and (e) recovering at least a portion of the C₂ hydrocarbons and/or at least a portion of the synthesis gas (e.g., simultaneous recovery of H₂ and CO products) from the product mixture.

In an embodiment, a method for producing ethylene can comprise (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 1,000° C., and wherein the reactor is characterized by a residence time of from about 1 millisecond to about 100 milliseconds in the catalyst bed; (b) wherein isothermal reactor conditions minimize hot spots formation in the catalyst bed, thereby decreasing an incidence of deep oxidation reactions, when compared to an incidence of deep oxidation reactions in an otherwise similar oxidative coupling of CH₄ reaction conducted under non-isothermal conditions; (c) allowing at least a portion of the reactant mixture to contact the catalyst and react via an oxidative coupling of CH₄ reaction to form a product mixture under isothermal conditions, wherein the product mixture comprises C₂ hydrocarbons and synthesis gas, wherein the C₂ hydrocarbons comprise ethylene and ethane, wherein the product mixture is characterized by an ethylene/ethane molar ratio of from about 0.5:1 to about 20:1, wherein the synthesis gas comprises hydrogen (H₂) and carbon monoxide (CO), and wherein the product mixture is characterized by a H₂/CO molar ratio of from about 0.5:1 to about 2.5:1; (d) recovering at least a portion of the product mixture from the reactor; and (e) recovering at least a portion of the C₂ hydrocarbons and/or at least a portion of the synthesis gas from the product mixture.

In an embodiment, a method for producing ethylene can comprise (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 950° C., and wherein the reactor is characterized by a residence time of from about 15 millisecond to about 25 milliseconds in the catalyst bed, and wherein isothermal reactor conditions minimize hot spots formation within the reactor; (b) allowing at least a portion of the reactant mixture to contact the catalyst and react via an oxidative coupling of CH₄ reaction to form a product mixture, wherein the product mixture comprises ethylene, and wherein a selectivity to ethylene is increased by equal to or greater than about 50% when compared to a selectivity of an otherwise similar oxidative coupling of CH₄ reaction conducted under non-isothermal conditions; and (c) recovering at least a portion of the product mixture from the reactor. In such embodiment, the method for producing ethylene can further comprise minimizing deep oxidation of methane to CO₂, wherein the product mixture comprises less than about 10 mol % CO₂. In an embodiment, the product mixture comprises synthesis gas.

In an embodiment, a method for producing ethylene can comprise (a) introducing a reactant mixture to an isothermal reactor, wherein the reactant mixture comprises methane (CH₄) and oxygen (O₂), wherein the reactant mixture is characterized by a CH₄/O₂ molar ratio of from about 4:1 to about 8:1, wherein the isothermal reactor comprises a catalyst bed comprising a catalyst, wherein an isothermal reaction temperature in the catalyst bed is from about 800° C. to about 950° C., and wherein the reactor is characterized by a residence time of from about 10 millisecond to about 50 milliseconds in the catalyst bed; (b) allowing at least a portion of the reactant mixture to contact the catalyst and react via an oxidative coupling of CH₄ reaction to form a product mixture, wherein the product mixture comprises ethylene, and wherein a selectivity to ethylene is increased by equal to or greater than about 40% when compared to a selectivity of an otherwise similar oxidative coupling of CH₄ reaction conducted under non-isothermal conditions; (c) recovering at least a portion of the product mixture from the reactor; and (d) separating at least a portion of the ethylene from the product mixture by cryogenic distillation to yield recovered ethylene. In such embodiment, the product mixture comprises synthesis gas, wherein the synthesis gas can be separated from the product mixture by cryogenic distillation to yield recovered synthesis gas, and wherein the recovered synthesis gas can be further used for producing olefins.

In an embodiment, a method for producing olefins (e.g., ethylene) as disclosed herein can advantageously display improvements in one or more method characteristics when compared to an otherwise similar method conducted under non-isothermal conditions. In an embodiment, the method for producing olefins (e.g., ethylene) as disclosed herein can advantageously display an enhanced C₂₌ selectivity when compared to an otherwise similar method of producing olefin conducted under non-isothermal conditions. An overall increase in C₂₊ selectivity (owing in part to an increase in C₂₌ selectivity) can advantageously lead to a sum of methane conversion plus C₂₊ selectivity of greater than about 100%.

In an embodiment, a method for producing olefins (e.g., ethylene) as disclosed herein can advantageously provide for minimizing hot spots formation in the reactor when compared to an otherwise similar method conducted under non-isothermal conditions. Additional advantages of the methods for the production of olefins (e.g., ethylene) as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

Oxidative coupling of methane (OCM) reactions were conducted under isothermal conditions as follows. A mixture of methane and oxygen along with an internal standard, an inert gas (neon) were fed to a small quartz reactor with an internal diameter (I.D.) of 2.3 mm, which was located in a fluidized sand bath (BFS high temperature furnace, which is commercially available from Techne Calibration). A catalyst (e.g., catalyst bed) loading was 50 mg, and total flow rates of gases corresponded to a residence time of 18 ms in the catalyst bed. The reactor was first heated to a desired temperature under an inert gas flow and then a desired gas mixture was fed to the reactor. For Run #1, a catalyst loading of 100 mg was used in a 4 mm I.D. quartz reactor tube in a traditional clamshell furnace. The OCM reaction was conducted in a similar way as described above for isothermal condition.

Selectivities and conversions were calculated as outlined in equations (1)-(7), and the data are displayed in Tables 1 and 2. All data in Tables 1 and 2 were acquired by using the same catalyst, Na₂WO₄—Mn—O/SiO₂.

TABLE 1

| | Run #1 | Run #2 |
|---|---|---|
| Temperature, ° C. | 750 | 877 |
| Residence time, ms | 54 | 18 |
| CH4/O2 ratio | 7.4 | 7.4 |
| % CH₄ Conversion | 19.4 | 20.1 |
| % O₂ Conversion | 99.7 | 100.0 |
| ‘C’ Selectivities, % | | |
| C₂= | 39.6 | 59.8 |
| C₂ | 33.0 | 10.9 |
| C₃= | 4.2 | 7.6 |
| C₃ | 1.9 | 0.1 |
| C₂+ | 78.6 | 78.4 |
| CO | 6.2 | 9.8 |
| CO₂ | 15.2 | 11.8 |
| H₂/CO | 0.4 | 1.8 |
| C₂=/C₂ | 1.2 | 5.5 |
| ‘C’ BALANCE, % | 99.3 | 100.2 |

TABLE 2

Temperature, ° C.

| | | 877 | 900 |
|---|---|---|---|
| | 852 | (Run #2 of Table 1) | (Run #3) |
| Residence time, ms | 18 | 18 | 18 |
| CH4/O2 ratio | 7.4 | 7.4 | 7.4 |
| % CH₄ Conversion | 18.6 | 20.1 | 18.7 |
| % O₂ Conversion | 92.7 | 100.0 | 100.0 |
| ‘C’ Selectivities, % | | | |
| C₂= | 53.1 | 59.8 | 61.9 |
| C₂ | 12.9 | 10.9 | 5.8 |
| C₃= | 6.1 | 7.6 | 7.6 |
| C₃ | 0.3 | 0.1 | 0.0 |
| C₂+ | 72.2 | 78.4 | 75.2 |
| CO | 17.2 | 9.8 | 14.9 |
| CO₂ | 10.6 | 11.8 | 9.9 |
| H₂/CO | 1.5 | 1.8 | 1.9 |
| C₂=/C₂ | 4.1 | 5.5 | 10.8 |
| ‘C’ BALANCE, % | 97.9 | 100.2 | 99.1 |

The data in Table 1 show that the C₂₌ selectivity can be enhanced by using an isothermal reactor at a higher temperature and a shorter residence time (Run #2). A special feature of experiments described in Example 1 of the current disclosure is that isothermal conditions drastically reduce or eliminate formation of hot spots in the catalyst bed, which in turn leads to achieving high selectivity.

The data in Table 2 show that for a CH₄/O₂ feed ratio of 7.4 and with a Na₂WO₄—Mn—O/SiO₂ catalyst, the olefin content is enhanced even further at higher temperatures, although the total C₂ selectivity is decreased. The very high C₂H₄/C₂H₆ ratio achieved in this experiment (e.g., Run #3 of Table 2), coupled with a high H₂/CO ratio represents a significant advantage of this experiment: (i) from an ethylene separation point of view; and (ii) for a syngas application for a Fisher-Tropsch process, which requires a H₂/CO ratio close to 2.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### ADDITIONAL DISCLOSURE

A first aspect, which is a method for producing olefins comprising (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 1,000° C., and wherein the reactor is characterized by a residence time of from about 1 millisecond to about 100 milliseconds in the catalyst bed; (b) wherein isothermal reactor conditions minimize hot spots formation in the catalyst bed, thereby decreasing an incidence of deep oxidation reactions, when compared to an incidence of deep oxidation reactions in an otherwise similar oxidative coupling of CH₄ reaction conducted under non-isothermal conditions; (c) allowing at least a portion of the reactant mixture to contact the catalyst and react via an oxidative coupling of CH₄ reaction to form a product mixture under isothermal conditions, wherein the product mixture comprises C₂₊ hydrocarbons and synthesis gas, wherein the C₂₊ hydrocarbons comprise olefins and paraffins, wherein the C₂₊ hydrocarbons comprise C₂ hydrocarbons and C₃ hydrocarbons, wherein the product mixture is characterized by an olefin/paraffin molar ratio of from about 0.5:1 to about 20:1, wherein the synthesis gas comprises hydrogen (H₂) and carbon monoxide (CO), and wherein the product mixture is characterized by a H₂/CO molar ratio of from about 0.2:1 to about 2.5:1; (d) recovering at least a portion of the product mixture from the reactor; and (e) recovering at least a portion of the C₂ hydrocarbons and/or at least a portion of the synthesis gas from the product mixture.

A second aspect, which is the method of the first aspect, wherein the isothermal reaction temperature in the catalyst bed is less than about 900° C.

A third aspect, which is the method of any one of the first and the second aspects, wherein the isothermal reactor comprises a reactor vessel located inside a fluidized sand bath reactor.

A fourth aspect, which is the method of the third aspect, wherein the isothermal conditions are provided by fluidization of heated microspheres around the isothermal reactor comprising the catalyst bed, wherein the microspheres are heated at a temperature of from about 725° C. to about 1,000° C., and wherein the microspheres comprise sand, metal oxides, quartz sand, aluminum oxide, silicon carbide, or combinations thereof.

A fifth aspect, which is the method of any one of the first through the fourth aspects, wherein the isothermal reactor comprises a fixed bed reactor.

A sixth aspect, which is the method of any one of the first through the fifth aspects, wherein the reactant mixture is characterized by a CH₄/O₂ molar ratio of from about 2:1 to about 40:1.

A seventh aspect, which is the method of any one of the first through the sixth aspects, wherein the isothermal reactor is characterized by a pressure of from about ambient pressure to about 500 psig.

An eighth aspect, which is the method of any one of the first through the seventh aspects, wherein the isothermal reactor is characterized by a weight hourly space velocity of from about 3,600 h⁻¹ to about 36,000 h⁻¹.

A ninth aspect, which is the method of any one of the first through the eighth aspects, wherein the reactant mixture further comprises a diluent.

A tenth aspect, which is the method of the ninth aspect, wherein the diluent contributes to isothermal conditions of reactor.

An eleventh aspect, which is the method of any one of the first through the tenth aspects, wherein the diluent comprises water, nitrogen, inert gases, or combinations thereof.

A twelfth aspect, which is the method of any one of the first through the eleventh aspects, wherein the catalyst catalyzes a high temperature oxidative conversion of CH₄ to C₂ hydrocarbons and synthesis gas.

A thirteenth aspect, which is the method of any one of the first through the twelfth aspects, wherein the catalyst comprises basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; or combinations thereof.

A fourteenth aspect, which is the method of any one of the first through the thirteenth aspects, wherein the catalyst comprises CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Sr—La/CeO₂, Sr—Ce/La₂O₃, Na—Mn—La₂O₃/Al₂O₃, Na—Mn—O/SiO₂, Na₂WO₄—Mn/SiO₂, Na₂WO₄—Mn—O/SiO₂, or combinations thereof.

A fifteenth aspect, which is the method of any one of the first through the fourteenth aspects, wherein the product mixture comprises coupling products, partial oxidation products, and unreacted methane.

A sixteenth aspect, which is the method of any one of the first through the fifteenth aspects, wherein the product mixture comprise C₂H₄, C₂H₆, CH₄, CO, H₂, CO₂ and H₂O.

A seventeenth aspect, which is the method of any one of the first through the sixteenth aspects, wherein a selectivity to C₂₊ hydrocarbons and synthesis gas is from about 60% to about 99%.

An eighteenth aspect, which is the method of any one of the first through the seventeenth aspects, wherein a methane conversion is from about 10% to about 45%.

A nineteenth aspect, which is the method of any one of the first through the eighteenth aspects, wherein the C₂ hydrocarbons comprise ethylene and ethane.

A twentieth aspect, which is the method of the nineteenth aspect, wherein a molar ratio of ethylene to ethane is from about 0.5:1 to about 20:1.

A twenty-first aspect, which is the method of any one of the first through the twentieth aspects, wherein the C₃ hydrocarbons comprise propylene and propane.

A twenty-second aspect, which is the method of the twenty-first aspect, wherein a molar ratio of propylene to propane is from about 0.5:1 to about 50:1.

A twenty-third aspect, which is the method of any one of the first through the twenty-second aspects, wherein a selectivity to C₂ hydrocarbons is from about 55% to about 95%.

A twenty-fourth aspect, which is the method of any one of the first through the twenty-third aspects, wherein a selectivity to ethylene is from about 20% to about 80%.

A twenty-fifth aspect, which is the method of any one of the first through the twenty-fourth aspects, wherein a selectivity to C₂₊ hydrocarbons is from about 60% to about 95%.

A twenty-sixth aspect, which is the method of any one of the first through the twenty-fifth aspects, wherein equal to or greater than about 5 mol % of the reactant mixture is converted to olefins.

A twenty-seventh aspect, which is the method of any one of the first through the twenty-sixth aspects, wherein equal to or greater than about 5 mol % of the reactant mixture is converted to ethylene.

A twenty-eighth aspect, which is the method of any one of the first through the twenty-seventh aspects, wherein equal to or greater than about 10 mol % of the reactant mixture is converted to C₂ hydrocarbons.

A twenty-ninth aspect, which is the method of any one of the first through the twenty-eighth aspects, wherein equal to or greater than about 12 mol % of the reactant mixture is converted to C₂₊ hydrocarbons.

A thirtieth aspect, which is the method of any one of the first through the twenty-ninth aspects, wherein equal to or greater than about 5 mol % of the reactant mixture is converted to synthesis gas.

A thirty-first aspect, which is the method of any one of the first through the thirtieth aspects, wherein a selectivity to CO is from about 5% to about 25%.

A thirty-second aspect, which is the method of any one of the first through the thirty-first aspects, wherein at least a portion of the synthesis gas is separated from the product mixture to yield recovered synthesis gas.

A thirty-third aspect, which is the method of any one of the first through the thirty-second aspects, wherein at least a portion of the synthesis gas is separated from the product mixture by cryogenic distillation.

A thirty-fourth aspect, which is the method of the thirty-second aspect, wherein at least a portion of the recovered synthesis gas is further converted to olefins.

A thirty-fifth aspect, which is the method of the fifteenth aspect, wherein at least a portion of the synthesis gas and at least a portion of the unreacted methane are separated from the product mixture to yield a recovered synthesis gas mixture.

A thirty-sixth aspect, which is the method of the thirty-fifth aspect, wherein at least a portion of the recovered synthesis gas mixture is further converted to olefins.

A thirty-seventh aspect, which is the method of any one of the first through the thirty-sixth aspects, wherein at least a portion of the recovered synthesis gas mixture is further converted to liquid hydrocarbons by a Fischer-Tropsch process.

A thirty-eighth aspect, which is the method of any one of the first through the thirty-seventh aspects, wherein at least a portion of the recovered synthesis gas mixture is further used as fuel to generate power.

A thirty-ninth aspect, which is the method of any one of the first through the thirty-eighth aspects, wherein at least a portion of the C₂₊ hydrocarbons is separated from the product mixture to yield recovered C₂₊ hydrocarbons.

A fortieth aspect, which is the method of the thirty-ninth aspect, wherein at least a portion of the recovered C₂₊ hydrocarbons is used for ethylene production.

A forty-first aspect, which is the method of the fortieth aspect further comprising separating at least a portion of the ethylene from the recovered C₂₊ hydrocarbons to yield recovered ethylene.

A forty-second aspect, which is the method of any one of the first through the forty-first aspects further comprising converting at least a portion of the recovered C₂₊ hydrocarbons to ethylene.

A forty-third aspect, which is the method of any one of the first through the forty-second aspects, wherein at least a portion of the unreacted methane is separated from the product mixture to yield recovered methane.

A forty-fourth aspect, which is the method of the forty-third aspect, wherein at least a portion of the recovered methane is recycled to the reactant mixture.

A forty-fifth aspect, which is the method of any one of the first through the forty-fourth aspects, wherein at least a portion of the recovered synthesis gas mixture is further converted to methane via a methanation process.

A forty-sixth aspect, which is the method of any one of the first through the forty-fifth aspects, wherein at least a portion of the unreacted methane is recovered and recycled to the reactant mixture.

A forty-seventh aspect, which is a method for producing olefins comprising (a) introducing a reactant mixture to an isothermal reactor, wherein the reactant mixture comprises methane (CH₄) and oxygen (O₂), wherein the isothermal reactor comprises a catalyst bed comprising a catalyst, wherein an isothermal reaction temperature in the catalyst bed is from about 750° C. to about 950° C., and wherein the reactor is characterized by a residence time of from about 1 millisecond to about 100 milliseconds in the catalyst bed, and wherein isothermal reactor conditions minimize hot spots formation within the reactor; (b) allowing at least a portion of the reactant mixture to contact the catalyst and react via an oxidative coupling of CH₄ reaction to form a product mixture, wherein the product mixture comprises olefins, and wherein a selectivity to olefins is increased by equal to or greater than about 10% when compared to a selectivity of an otherwise similar oxidative coupling of CH₄ reaction conducted under non-isothermal conditions; and (c) recovering at least a portion of the product mixture from the reactor.

A forty-eighth aspect, which is the method of the forty-seventh aspect further comprising minimizing deep oxidation of methane to carbon dioxide (CO₂).

A forty-ninth aspect, which is the method of any one of the forty-seventh and the forty-eighth aspects, wherein the product mixture comprises less than about 10 mol % carbon dioxide (CO₂).

A fiftieth aspect, which is the method of any one of the forty-seventh through the forty-ninth aspects, wherein the product mixture comprises synthesis gas.

A fifty-first aspect, which is a method for producing ethylene comprising (a) introducing a reactant mixture to an isothermal reactor, wherein the reactant mixture comprises methane (CH₄) and oxygen (O₂), wherein the reactant mixture is characterized by a CH₄/O₂ molar ratio of from about 4:1 to about 8:1, wherein the isothermal reactor comprises a catalyst bed comprising a catalyst, wherein an isothermal reaction temperature in the catalyst bed is from about 800° C. to about 900° C., and wherein the reactor is characterized by a residence time of from about 10 millisecond to about 50 milliseconds in the catalyst bed; (b) allowing at least a portion of the reactant mixture to contact the catalyst and react via an oxidative coupling of CH₄ reaction to form a product mixture, wherein the product mixture comprises ethylene, and wherein a selectivity to ethylene is increased by equal to or greater than about 40% when compared to a selectivity of an otherwise similar oxidative coupling of CH₄ reaction conducted under non-isothermal conditions; (c) recovering at least a portion of the product mixture from the reactor; and (d) separating at least a portion of the ethylene from the product mixture by cryogenic distillation to yield recovered ethylene.

A fifty-second aspect, which is the method of the fifty-first aspect, wherein the product mixture comprises synthesis gas, and wherein the synthesis gas is separated from the product mixture by cryogenic distillation to yield recovered synthesis gas.

While aspects of the disclosure have been shown and described, modifications thereof can be made without departing from the spirit and teachings of the invention. The aspects and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an aspect of the present invention. Thus, the claims are a further description and are an addition to the detailed description of the present invention. The disclosures of all patents, patent applications, and publications cited herein are hereby incorporated by reference.

What is claimed is:

1. A method for producing olefins comprising:
(a) introducing a reactant mixture to an isothermal reactor, wherein the reactant mixture comprises methane (CH4) and oxygen (O2), wherein the isothermal reactor comprises a catalyst bed comprising a catalyst, wherein an isothermal reaction temperature in the catalyst bed is from about 750° C. to about 1,000° C., and wherein the reactor is characterized by a residence time of from about 1 millisecond to about 100 milliseconds in the catalyst bed;
(b) wherein isothermal reactor conditions minimize hot spots formation in the catalyst bed, thereby decreasing an incidence of deep oxidation reactions, when compared to an incidence of deep oxidation reactions in an otherwise similar oxidative coupling of CH4 reaction conducted under non-isothermal conditions;
(c) allowing at least a portion of the reactant mixture to contact the catalyst and react via an oxidative coupling of CH4 reaction to form a product mixture under isothermal conditions, wherein the product mixture comprises C2+ hydrocarbons and synthesis gas, wherein the C2+ hydrocarbons comprise olefins and paraffins, wherein the C2+ hydrocarbons comprise C2 hydrocarbons and C3 hydrocarbons, wherein the product mixture is characterized by an olefin/paraffin molar ratio of from about 0.5:1 to about 20:1, wherein the synthesis gas comprises hydrogen (H2) and carbon monoxide (CO), and wherein the product mixture is characterized by a H2/CO molar ratio of from about 0.2:1 to about 2.5:1;
(d) recovering at least a portion of the product mixture from the reactor; and
(e) recovering at least a portion of the C2 hydrocarbons and/or at least a portion of the synthesis gas from the product mixture.

2. The method of claim 1, wherein the isothermal reaction temperature in the catalyst bed is less than about 900° C.

3. The method of claim 1, wherein the isothermal reactor comprises a reactor vessel located inside a fluidized sand bath reactor.

4. The method of claim 3, wherein the isothermal conditions are provided by fluidization of heated microspheres around the isothermal reactor comprising the catalyst bed, wherein the microspheres are heated at a temperature of from about 725° C. to about 1,000° C., and wherein the microspheres comprise sand, metal oxides, quartz sand, aluminum oxide, silicon carbide, or combinations thereof.

5. The method of claim 1, wherein a selectivity to C2+ hydrocarbons and synthesis gas is from about 60% to about 99%.

6. The method of claim 1, wherein a methane conversion is from about 10% to about 45%.

7. The method of claim 1, wherein the C2 hydrocarbons comprise ethylene and ethane and wherein a molar ratio of ethylene to ethane is from about 0.5:1 to about 20:1.

8. The method of claim 1, wherein the C3 hydrocarbons comprise propylene and propane and wherein a molar ratio of propylene to propane is from about 0.5:1 to about 50:1.

9. The method of claim 1, wherein a selectivity to C2 hydrocarbons is from about 55% to about 95%.

10. The method of claim 1, wherein a selectivity to ethylene is from about 20% to about 80%.

11. The method of claim 1, wherein a selectivity to C2+ hydrocarbons is from about 60% to about 95%.

12. The method of claim 1, wherein equal to or greater than about 5 mol % of the reactant mixture is converted to olefins.

13. The method of claim 1, wherein equal to or greater than about 5 mol % of the reactant mixture is converted to synthesis gas.

14. The method of claim 1, wherein a selectivity to CO is from about 5% to about 25%.

15. A method for producing olefins comprising:
(a) introducing a reactant mixture to an isothermal reactor, wherein the reactant mixture comprises methane (CH4) and oxygen (O2), wherein the isothermal reactor comprises a catalyst bed comprising a catalyst, wherein an isothermal reaction temperature in the catalyst bed is from about 750° C. to about 950° C., and wherein the reactor is characterized by a residence time of from about 1 millisecond to about 100 milliseconds in the catalyst bed, and wherein isothermal reactor conditions minimize hot spots formation within the reactor;
(b) allowing at least a portion of the reactant mixture to contact the catalyst and react via an oxidative coupling of CH4 reaction to form a product mixture, wherein the product mixture comprises olefins, and wherein a selectivity to olefins is increased by equal to or greater than about 10% when compared to a selectivity of an otherwise similar oxidative coupling of CH4 reaction conducted under non-isothermal conditions; and
(c) recovering at least a portion of the product mixture from the reactor.

16. The method of claim 15 further comprising minimizing deep oxidation of methane to carbon dioxide (CO2).

17. The method of claim 15, wherein the product mixture comprises less than about 10 mol % carbon dioxide (CO2).

18. The method of claim 15, wherein the product mixture comprises synthesis gas.

19. A method for producing ethylene comprising:
(a) introducing a reactant mixture to an isothermal reactor, wherein the reactant mixture comprises methane (CH4) and oxygen (O2), wherein the reactant mixture is characterized by a CH4/O2 molar ratio of from about 4:1 to about 8:1, wherein the isothermal reactor comprises a catalyst bed comprising a catalyst, wherein an isothermal reaction temperature in the catalyst bed is from about 800° C. to about 900° C., and wherein the reactor is characterized by a residence time of from about 10 millisecond to about 50 milliseconds in the catalyst bed;
(b) allowing at least a portion of the reactant mixture to contact the catalyst and react via an oxidative coupling of CH4 reaction to form a product mixture, wherein the product mixture comprises ethylene, and wherein a selectivity to ethylene is increased by equal to or greater than about 40% when compared to a selectivity of an otherwise similar oxidative coupling of CH4 reaction conducted under non-isothermal conditions;
(c) recovering at least a portion of the product mixture from the reactor; and
(d) separating at least a portion of the ethylene from the product mixture by cryogenic distillation to yield recovered ethylene.

20. The method of claim 19, wherein the product mixture comprises synthesis gas, and wherein the synthesis gas is separated from the product mixture by cryogenic distillation to yield recovered synthesis gas.
